(19) United States
(12) Patent Application Publication
(10) Pub. No.: US 2026/0331232 A1
(43) Pub. Date: Oct. 22, 2026

(54) CHEMISTRY INFORMED MACHINE LEARNING FOR REACTIVE TRANSPORT SIMULATIONS OF SUBSURFACE SYSTEMS

(71) Applicant: Schlumberger Technology Corporation, Sugar Land, TX (US)

(72) Inventor: Soham Sheth, Abingdon (GB)

(21) Appl. No.: 19/640,010

(22) Filed: Apr. 6, 2026

Related U.S. Application Data
(60) Provisional application No. 63/790,369, filed on Apr. 17, 2025.

(51) Int. Cl. G16C 20/70; E21B 41/00; G16C 20/10

(52) U.S. Cl. CPC G16C 20/70; E21B 41/005; G16C 20/10; E21B 2200/22

(57) ABSTRACT

A method includes providing, as an input to a neural network (NN) model, one or more parameters associated with a geochemical process. The method also includes receiving, as an output of the NN model, a predicted state of the geochemical process. Further, the method includes providing, as an input to a physics-based model, the predicted state of the geochemical process. Further still, the process includes determining, as an output of the physics-based model, a discrepancy associated with the predicted state of the geochemical process. Even further, the process includes updating the NN model based on the discrepancy.

### CROSS-REFERENCE TO RELATED APPLICATION

This application claims benefit of U.S. Provisional Patent Application No. 63/790,369, filed on Apr. 17, 2025, which is hereby incorporated by reference in its entirety for all purposes.

### BACKGROUND

The present disclosure generally relates to techniques for simulating geochemical processes in large-scale sub surfaces. More specifically, the present disclosure relates to utilizing a physics-based model in conjunction with a neural network (NN) model or a machine-learning (ML) model to refine the NN model or ML model and control operation of oil and gas equipment.

The use of reactive-transport models for simulating geochemical processes in large-scale subsurface systems is important in the formulation of strategies for geologic carbon sequestration, hydrogen storage, and various others engineering applications. Key geochemical processes include mineral precipitation and dissolution, redox and biogeochemical reactions, and dissolution. When CO₂ is injected into the subsurface, the CO₂ disturbs existing geochemical equilibrium. In turn, this can promote conditions that induce mineral dissolution, salt precipitation near the wellbore due to water vaporization, and possible dolomitization. In some cases, these geochemical effects are significant. As such, the accurate representation of geochemical effects is essential.

Fundamentally, when modeling reactive transport simulations, mass conservation at the transport level applies to each chemical species in the fluid phases. Therefore, a transport equation is needed for each chemical species, which makes the simulation computationally expensive, especially when geochemical models comprising dozens of chemical species are needed to thermodynamically represent an aqueous solution.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

A method includes providing, as an input to a neural network (NN) model, one or more parameters associated with a geochemical process. The method also includes receiving, as an output of the NN model, a predicted state of the geochemical process. Further, the method includes providing, as an input to a physics-based model, the predicted state of the geochemical process. Further still, the process includes determining, as an output of the physics-based model, a discrepancy associated with the predicted state of the geochemical process. Even further, the process includes updating the NN model based on the discrepancy.

A tangible, non-transitory, machine-readable medium comprising instructions that, when executed by a processor, cause the processor to perform operations comprising providing, as an input to a neural network (NN) model, one or more parameters associated with a geochemical process. The operations also include receiving, as an output of the NN model, a predicted state of the geochemical process. Further, the operations include providing, as an input to a physics-based model, the predicted state of the geochemical process. Further still, the operations include determining, as an output of the physics-based model, a discrepancy associated with the predicted state of the geochemical process. Even further, the operations include updating the NN model based on the discrepancy.

A system includes one or more downhole tools. The system also includes a control system, comprising one or more processors, wherein the control system is configured to perform operations comprising providing, as an input to a neural network (NN) model, one or more parameters associated with a geochemical process. The operations also include receiving, as an output of the NN model, a predicted state of the geochemical process. Further, the operations include providing, as an input to a physics-based model, the predicted state of the geochemical process. Further still, the operations include determining, as an output of the physics-based model, a discrepancy associated with the predicted state of the geochemical process. Even further, the operations include controlling operation of oil and gas equipment based on the discrepancy.

The brief summary presented above is intended only to familiarize the reader with certain aspects and contexts of embodiments of the present disclosure without limitation to the claimed subject matter.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a block diagram of a computing system, in accordance with an embodiment of the present disclosure;

FIG. 2 shows a downhole tool that may measure properties of one or more fluids within a well, in accordance with an embodiment of the present disclosure;

FIG. 3 is a schematic diagram of a training model used to generate an updated NN model using a physic-based model, in accordance with an embodiment of the present disclosure; and

FIG. 4 is a flow diagram of an example method for refining a NN model, in accordance with an embodiment of the present disclosure.

### DETAILED DESCRIPTION

Certain embodiments commensurate in scope with the present disclosure are summarized below. These embodiments are not intended to limit the scope of the disclosure, but rather these embodiments are intended only to provide a brief summary of certain disclosed embodiments. Indeed, the present disclosure may encompass a variety of forms that may be similar to or different from the embodiments set forth below.

As used herein, the term “coupled” or “coupled to” may indicate establishing either a direct or indirect connection (e.g., where the connection may not include or include intermediate or intervening components between those coupled), and is not limited to either unless expressly referenced as such. The term “set” may refer to one or more items. Wherever possible, like or identical reference numerals are used in the figures to identify common or the same elements. The figures are not necessarily to scale and certain features and certain views of the figures may be shown exaggerated in scale for purposes of clarification.

As used herein, the terms “inner” and “outer”; “up” and “down”; “upper” and “lower”; “upward” and “downward”; “above” and “below”; “inward” and “outward”; and other like terms as used herein refer to relative positions to one another and are not intended to denote a particular direction or spatial orientation. The terms “couple,” “coupled,” “connect,” “connection,” “connected,” “in connection with,” and “connecting” refer to “in direct connection with” or “in connection with via one or more intermediate elements or members.”

Furthermore, when introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment,” “an embodiment,” or “some embodiments” of the present disclosure are not intended to be interpreted as excluding the existence of additional embodiments that also incorporate the recited features. Furthermore, the phrase A “based on” B is intended to mean that A is at least partially based on B. Moreover, unless expressly stated otherwise, the term “or” is intended to be inclusive (e.g., logical OR) and not exclusive (e.g., logical XOR). In other words, the phrase A “or” B is intended to mean A, B, or both A and B.

When modeling reactive transport simulations, mass conservation at the transport level applies to each chemical species in the fluid phases. Therefore, a transport equation is needed for each chemical species, which makes the simulation computationally expensive, especially when geochemical models comprising dozens of chemical species are needed to thermodynamically represent an aqueous solution. Machine learning models can help accelerate computations and could provide accurate predictions. In many cases, when only data is used to train these models, the predictions may show chemical inconsistencies.

Accordingly, the present disclosure relates to techniques for training and refining machine-learning models (e.g., ML models, NN models) using a physics-based model. For example, one or more processors or other computing device having a process may utilize a NN model to generate a predicted state of a geochemical process based on one or more input parameters related to the geochemical system or process. The geochemical process could be a fluid injection process into a downhole reservoir. One or more input parameters related to the geochemical process may include an amount of the fluid to be injected or actively being injected, a pressure within the downhole reservoir, a temperature within the downhole reservoir, or a solubility of the fluid within certain rocks of the downhole reservoir. The NN may provide an output that is predicted state of the geochemical process. For example, the predicted state may indicate a predicted pressure, predicted temperature, or other parameter, after the fluid is injected for a time period. To improve the accuracy of the NN model, the processor may provide the predicted state and the one or more input parameters to a physics-based model.

The physics-based model utilizes mathematical relationships in chemistry, physics, and geophysics to provide a predicted state. In some embodiments, the physics-based model may use one or more loss functions related to a geochemical process, such as Gibbs free energy, the law of mass action, equations related to chemical potential, or a chemical equation related to the products and reactants. In this way, the predicted state provided or otherwise output by the physics-model is constrained by these mathematical relationships. Then, the processor may compare the predicted state (e.g., a first predicted state) from the NN model (e.g., a first model) and a predicted state (e.g., a second predicted state) determined by physics-based model (e.g., a second model) and determine a discrepancy between the two predicted states. The processor may provide the discrepancy to the NN model as feedback. In turn, the NN model may use the discrepancy to update or otherwise refine the NN model. Accordingly, the disclosed techniques may increase the accuracy of certain ML models (e.g., NN models) that may be relatively inaccurate at extrapolating to determine predicted future states.

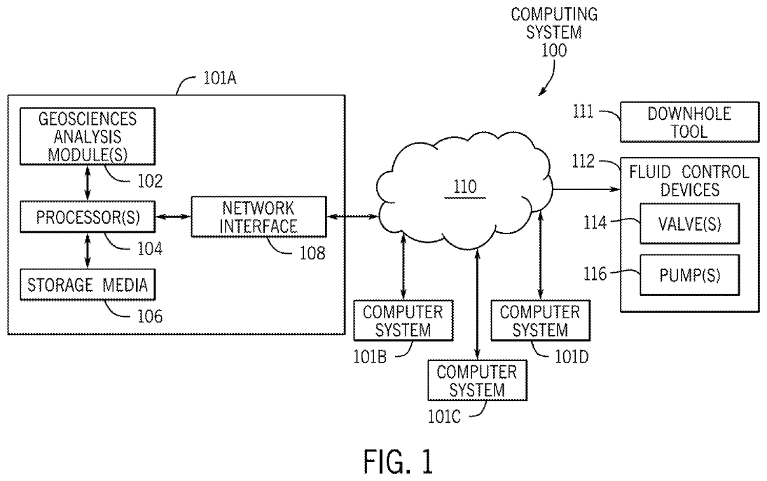

FIG. 1 depicts an example computing system 100 in accordance with some embodiments. The computing system 100 can be an individual computer system 101A or an arrangement of distributed computer systems. The computer system 101A includes one or more geosciences analysis modules 102 that are configured to perform various tasks according to some embodiments, such as one or more methods disclosed herein. To perform these various tasks, geosciences analysis module 102 executes independently, or in coordination with, one or more processors 104, which is (or are) connected to one or more storage media 106. The processor(s) 104 is (or are) also connected to a network interface 108 to allow the computer system 101A to communicate over a data network 110 with one or more additional computer systems and/or computing systems, such as 101B, 101C, and/or 101D (note that computer systems 101B, 101C and/or 101D may or may not share the same architecture as computer system 101A, and may be located in different physical locations, e.g., computer systems 101A and 101B may be on a ship underway on the ocean, while in communication with one or more computer systems such as 101C and/or 101D that are located in one or more data centers on shore, other ships, and/or located in varying countries on different continents). Note that data network 110 may be a private network, it may use portions of public networks, it may include remote storage and/or applications processing capabilities (e.g., cloud computing).

A processor can include a microprocessor, microcontroller, processor module or subsystem, programmable integrated circuit, programmable gate array, or another control or computing device.

The storage media 106 can be implemented as one or more computer-readable or machine-readable storage media. Note that while in the example embodiment of FIG. 1 storage media 106 is depicted as within computer system 101A, in some embodiments, storage media 106 may be distributed within and/or across multiple internal and/or external enclosures of computing system 101A and/or additional computing systems. Storage media 106 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories; magnetic disks such as fixed, floppy and removable disks; other magnetic media including tape; optical media such as compact disks (CDs) or digital video disks (DVDs), BluRays or any other type of optical media; or other types of storage devices. Note that the instructions discussed above can be provided on one computer-readable or machine-readable storage medium, or alternatively, can be provided on multiple computer-readable or machine-readable storage media distributed in a large system having possibly plural nodes and/or non-transitory storage means. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions, or located at a remote site from which machine-readable instructions can be downloaded over a network for execution.

It should be appreciated that computer system 101A is one example of a computing system, and that computer system 101A may have more or fewer components than shown, may combine additional components not depicted in the example embodiment of FIG. 1, and/or computer system 101A may have a different configuration or arrangement of the components depicted in FIG. 1. The various components shown in FIG. 1 may be implemented in hardware, software, or a combination of both, hardware and software, including one or more signal processing and/or application specific integrated circuits.

It should also be appreciated that while no user input/output peripherals are illustrated with respect to computer systems 101A, 101B, 101C, and 101D, many embodiments of computing system 100 include computer systems with keyboards, mice, touch screens, displays, etc. Some computer systems in use in computing system 100 may be desktop workstations, laptops, tablet computers, smartphones, server computers, etc.

Further, the steps in the processing methods described herein may be implemented by running one or more functional modules in information processing apparatus such as general purpose processors or application specific chips, such as ASICs, FPGAs, PLDs, or other appropriate devices. These modules, combinations of these modules, and/or their combination with general hardware are included within the scope of protection.

The processor 104 may control operation of one or more downhole tools 111 and/or fluid control devices 112 that are used in geochemical processes. The fluid control devices 112 may include valves 114 and/or pumps 116. As referred to herein, oil and gas equipment or hydrocarbon production equipment may refer to the one or more downhole tools 111 and/or fluid control devices 112

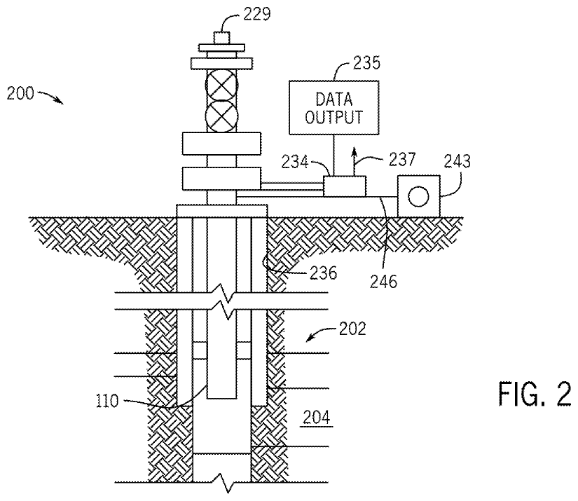

In some embodiments, the updated NN model may be used to control one or more hydrocarbon production equipment, such as valves, pumps, or downhole tools used during the geochemical process. FIG. 2 shows an example of a downhole tool 111 that may be controlled using an updated NN model. In particular, FIG. 2 illustrates a production operation being performed by downhole tool 111 (e.g., a production tool) deployed from a production unit or Christmas tree 229 and into completed wellbore 236 for drawing fluid (e.g., hydrocarbon fluid, oil, gas, etc.) from the downhole reservoirs into surface facilities 242. The fluid flows from reservoir 204 through perforations in the casing (not shown) and into production tool 100 in wellbore 236 and to surface facilities 242 via gathering network 246.

Sensors(S), such as gauges, may be positioned about oilfield 200 to collect data relating to various field operations as described previously. As shown, the sensor(S) may be positioned in production tool 100 or associated equipment, such as Christmas tree 229, gathering network 246, surface facility 242, and/or the production facility, to measure fluid parameters, such as fluid composition, flow rates, pressures, temperatures, and/or other parameters of the production operation.

Production may also include injection wells for added recovery. One or more gathering facilities may be operatively connected to one or more of the wellsites for selectively collecting downhole fluids from the wellsite(s).

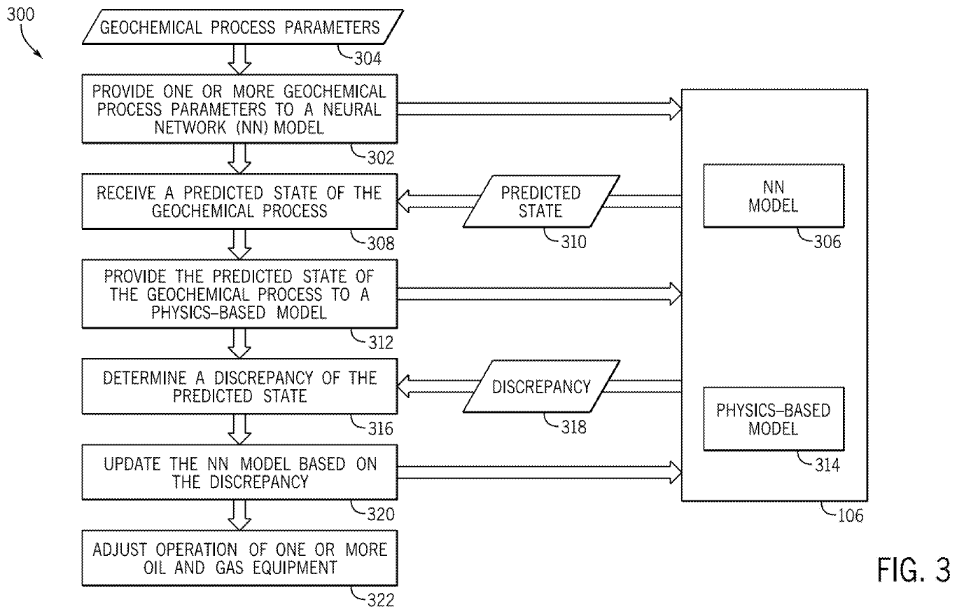

FIG. 3 shows an example of a process 300 for updating or refining a NN model using a physics-based module. Although the process 300 is described as being performed by the processor 104, it should be noted that any suitable computing system or controller with one or more processors may perform the process 300. In some embodiments, one or more of the blocks may be omitted or repeated.

At block 302, the processor 104 provides one or more geochemical process parameters 304 (e.g., input parameters) to the NN model 306. The one or more geochemical process parameters 304 relate to a geochemical process, such as a carbon capture and storage (CCS) process, a plume emission, hydrogen storage, geothermal processes, and other processes that relate to physical or chemical changes resulting from downhole conditions. As a specific example, the process parameters 304 may relate to a potential plume formation at a wellsite. Accordingly, the process parameters 304 may include pressure (P), temperature (T), concentration of one or more reagents within a reservoir, concentration of one or more reagents at a particular height in a potential plume (z_{c}), pH, and other parameters that may generally represent a present state of a geochemical process. As another example of a CCS process, the CCS process may relate to CCS precipitation within a geological formation. Accordingly, the process parameters 304 may relate to a volume of CO₂ to be injected into a geological formation, rock parameters related to the geological formation, a salinity of brine within the geological formation, and other parameters related to a capacity of the geological formation to store CO₂ and/or a likelihood of precipitation within the geological formation.

At block 308, the processor 104 receives a predicted state 310 of the geochemical process (e.g., an output) from the NN model 306. The predicted state 310 may include an estimated or otherwise modified value of the one or more geochemical process parameters 304 after a time interval (dt). As a specific example, the predicted state may indicate a change in the concentration of a plume at different heights over a time period. As another specific example, the predicted state may indicate a time period when precipitation resulting from a CCS process may occur. It is presently recognized that while an NN model may be accurate for interpolating states, the NN model 306 may be relatively inaccurate for extrapolating states (e.g., after the time interval dt). Thus, it may be beneficial to refine the NN model 306 by providing (e.g., via the processor 104) the predicted state 310 (e.g., predicted by the NN model 306) to a physics-based model.

At block 312, the processor 104 provides the predicted state 310 to the physics-based model 314. The physics-based model may calculate an additional predicted state using the predicted state 310 and the one or more geochemical process parameters 304 to the physics-based model 314. The output of the physics-based model 314 is a predicted state that may be different than the predicted state 310 generated by the NN model 306. That is, the predicted state may indicate a different concentration of the plume at different heights or a different time period when precipitation may occur.

To ensure that the outputs of the physics-based model 314 are accurate, the physics-based model 314 utilizes one or more loss functions related to geochemical processes to generate or provide an output predicted state. The loss functions may include Gibbs free energy, law of mass action, and other loss functions that are related to geochemical processes.

At block 316, the processor 104 determines a discrepancy 118 of the predicted state 310 using the physics-based model 314. In general, the processor 104 may compare the predicted state 310 output by the NN model 306 to the predicted state of the physics-based model 314 to determine the discrepancy. The discrepancy may be based on a root-mean-square error, mean absolute error, percentage error, and other error calculation for comparing outputs of models.

In some embodiments, the processor 104 may utilize sensor data acquired by one or more sensors (e.g., pressure sensors, temperature sensors, fluid flow sensors) to determine the discrepancy. For example, in an embodiment where the one or more process parameters relate to a potential or ongoing plume emission, the sensor may acquire pressure data related to the plume emission.

At block 320, the processor 104 updates or refines the NN model 306 based on the discrepancy 318. In some embodiments, the NN model 306 may be updated iteratively during a geochemical process. In any case, the NN model 306 may more accurately determine predicted states 310 (e.g., an updated predicted state or updated predicted states) that are similar to the output of the physics-based model 314. By using the NN model 306 as opposed to the physics-based model 314, outputs may be determined in substantially real time.

At block 322, the processor 104 adjusts operation of one or more oil and gas equipment using the NN model 306 (e.g., by outputting one or more controls signals). The oil and gas equipment may include the downhole 110 and/or fluid control devices 112 described in FIG. 1 or FIG. 2. In some embodiments, the processor 104 may utilize the updated or refined NN model (e.g., based on the NN model 306) to control operation of one or more hydrocarbon production equipment. For example, in an embodiment where the geochemical process is a CCS process, the processor 104 may control injection of CO₂ into a reservoir, such as by controlling a pressure, a temperature, and/or a flow rate of the CO₂ injection (e.g., via a control signal output to a fluid control device). In some embodiments, the processor 104 may control the CO₂ injection via increasing or decreasing a speed of a pump, opening or closing a valve, increasing or decreasing heat exchange in one or more heat exchangers, or any combination thereof, to maintain the CO₂ within a temperature range and a pressure range. In the example of a potential plume emission, the control action may be an adjustment to fluid control devices 112 to reduce the risk of the plume being emitted, prevent the plume from leaving a particular area, or prevent the plume from escaping from a vessel.

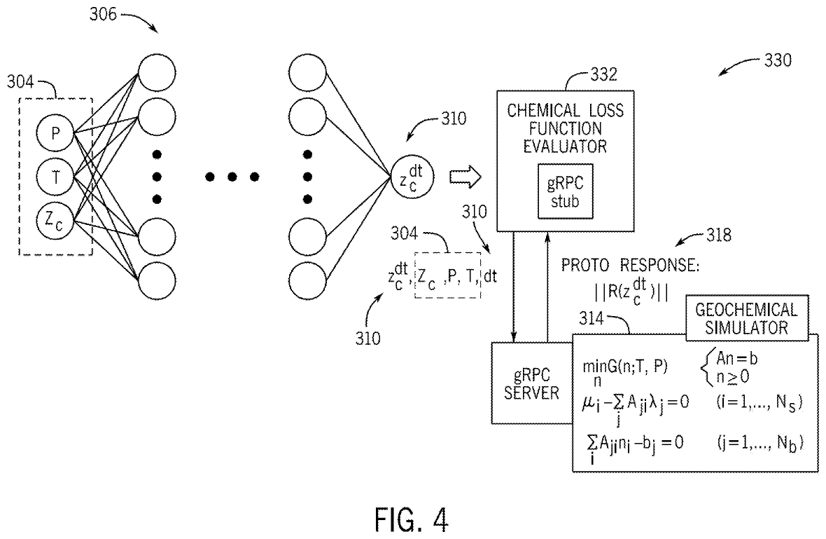

FIG. 4 shows an example of a schematic diagram of a training module 330 used to generate an updated NN model using a physic-based model. The training module 330 includes the NN model 306, the physics-based model 314, and a chemical loss function evaluator 332. In some embodiments, the chemical loss function evaluator 332 provides one or more loss functions and the derivatives for the ML model (e.g., NN model) to train, refine, or update. In some embodiments, the training module 330 may operate with unsupervised learning. In some embodiments, the training module 330 may operate using training data based on geochemical computations that serves as semi-supervised learning along with chemically consistent loss functions. Utilizing the training data generated based on geochemical calculations may result in improved convergence while training the ML models, and thus faster training, updating, or refining of the ML model.

In operation, the chemical loss function evaluator 332 receives the predicted state 310 output by the NN model 306. Then, the chemical loss function evaluator 332 may provide the one or more process parameters 304 to the physics-based model 314 (e.g., geochemical simulator), thereby generating an output predicted state by the physics-based model 314. The chemical loss function evaluator 332 may determine a discrepancy between outputs of the physics-based model 314 and the predicted state 310. The discrepancy may be provided to the NN model 306 to refine the NN model 306.

Several non-limiting examples of implementations of the training module 330 and the process 300 are described below. As a first example, the disclosed embodiments may be used for plume monitoring (e.g., potential plume). For example, relatively light hydrocarbons (e.g., methane) or other gaseous fluids may escape from a pressurized source. To reduce the risk of a plume being emitted, prevent the plume from leaving a particular area, or prevent the plume from escaping from a vessel, or otherwise mitigating risks associated with a plume (e.g., a potential plume), it may be advantageous to provide process parameters 304 including an initial pressure of the source, a volume of the source, a size the opening where the gaseous fluid is escaping or where the gaseous fluid may escape (e.g., a potential opening), an ambient temperature, an amount of the gaseous fluids initially within the source or any combination thereof. The NN model 306 may generate a first output (e.g., the predicted state 310) indicating a time evolution of the concentration of the gaseous fluids at various positions (e.g., heights) from the source. The physics-based model 314 may also receive the process parameters 304 and generate a second output (e.g., an additional predicted state) using Gibbs free energy as a loss function to constrain the predicted expansion of the gaseous fluid. The processor 104 may compare the first and second output and determine a discrepancy 318 that indicates the error in the predicted state 310. The NN model 306 may receive the discrepancy 318 and/or the second output as an input, thereby refining the NN model 306. Accordingly, the NN model 306 may be updated using feedback (e.g., the discrepancy 318) generated by the physics-based model 314.

As a second example, the disclosed embodiments may be used for preventing precipitation in a CCS process. In this example, it may be advantageous to provide process parameters 304 including an amount of CO₂ injected into a subterranean formation, a rate at which the CO₂ is injected into the subterranean formation, solubility of CO₂ in rocks within the subterranean formation, or any combination thereof. The NN model 306 may generate a first output (e.g., the predicted state 310) indicating a time evolution of the amount of CO₂ dissolved within the subterranean formation. The physics-based model 314 may also receive the process parameters 304 and generate a second output (e.g., an additional predicted state). The processor 104 may compare the first and second output and determine a discrepancy 318 that indicates the error in the predicted state 310. The NN model 306 may receive the discrepancy 318 and/or the second output as an input, thereby refining the NN model 306. Accordingly, the NN model 306 may be updated using feedback (e.g., the discrepancy 318) generated by the physics-based model 314. Further, the processor 304 may determine a schedule for injecting the CO₂ using the predicted state 310 (e.g., the corrected predicted state) from the NN model 306. For example, the processor 304 may determine a time period and/or a rate to inject the CO₂ to avoid or otherwise prevent precipitation.

The examples above are meant to be non-limiting, it should be noted that disclosed embodiments may also be used for applications such as logging-while-drilling applications. In this application, controlling the oil and gas equipment may include control drilling parameters, such a rotational speed of the drill string and/or drill bit, a flow rate of mud through a bottom hole assembly (BHA), a direction of drilling via a rotary steerable system (RSS) of the BHA, etc. As another example, the disclosed embodiments may be used during hydraulic fracturing operations to control a flow rate, composition, and/or pressure of the fracturing fluid being pumped into the wellbore. As another example, the disclosed embodiments may be used during chemical treatment operations, and control a flow rate, composition, pressure, temperature, and other injection parameters of a chemical treatment fluid.

The technical effect of the disclosed embodiments includes improving efficiency of controlling certain oil and gas operations. For example, the physics-based model may be more accurate with respect to determining predicted states (e.g., an extrapolated state or extrapolated states). However, the ML model may be more computationally efficient. However, by training the ML model using outputs of the physics-based model, the ML model may become more accurate at predicating states associated with geochemical processes. Thus, the ML model may be used to provide accurate information and in substantially real time. Further, the disclosed embodiments may allow for an ML model to train directly while minimizing the chemically informed loss function.

The subject matter described in detail above may be defined by one or more clauses, as set forth below.

A method includes providing, as an input to a neural network (NN) model, one or more parameters associated with a geochemical process. The method also includes receiving, as an output of the NN model, a predicted state of the geochemical process. Further, the method includes providing, as an input to a physics-based model, the predicted state of the geochemical process. Further still, the method includes determining, as an output of the physics-based model, a discrepancy associated with the predicted state of the geochemical process. Even further, the method includes updating the NN model based on the discrepancy.

The method of any preceding clause, further comprising determining an updated predicted state of the geochemical process using the updated NN model.

The method of any preceding clause, further comprising adjusting operation of one or more oil and gas equipment based on the updated NN model in real-time.

The method of any preceding clause, wherein the geochemical process is a carbon capture and storage (CCS) process.

The method of any preceding clause, wherein the predicted state is associated with CCS precipitation.

The method of any preceding clause, wherein the geochemical process relates to a plume emission.

The method of any preceding clause, wherein the physics-based model utilizes a minimization of Gibbs free energy as a loss function.

The method of any preceding clause, wherein the physics-based model utilizes law of mass action as a loss function.

A tangible, non-transitory, machine-readable medium comprising instructions that, when executed by a processor, cause the processor to perform operations comprising providing, as an input to a neural network (NN) model, one or more parameters associated with a geochemical process. The operations also include receiving, as an output of the NN model, a predicted state of the geochemical process. Further, the operations include providing, as an input to a physics-based model, the predicted state of the geochemical process. Further still, the operations include determining, as an output of the physics-based model, a discrepancy associated with the predicted state of the geochemical process. Even further, the operations include updating the NN model based on the discrepancy.

The tangible, non-transitory, machine-readable medium of any preceding clause, wherein the instructions, when executed, further cause the processor to perform the operations comprising controlling one or more oil and gas equipment based on the updated NN model.

The tangible, non-transitory, machine-readable medium of any preceding clause, wherein the one or more oil and gas equipment comprise fluid control devices.

The tangible, non-transitory, machine-readable medium of any preceding clause, wherein the instructions that cause the processor to control the one or more oil and gas equipment comprise adjusting a flow rate provided by the fluid control devices.

The tangible, non-transitory, machine-readable medium of any preceding clause, wherein the instructions that cause the processor to control the one or more oil and gas equipment comprise determining a time period to inject one or more fluids within a wellbore; and outputting a control signal that causes the fluid control devices to inject one or more fluids into a wellbore in accordance with the time period.

The tangible, non-transitory, machine-readable medium of any preceding clause, wherein the one or more parameters associated with the geochemical process relate to a concentration of a plume.

The tangible, non-transitory, machine-readable medium of any preceding clause, wherein the predicted state is an extrapolated state.

The tangible, non-transitory, machine-readable medium of any preceding clause, wherein the instructions that cause the processor to determine the discrepancy based on sensor data and an output of physics-based model.

A system includes one or more downhole tools. The system also includes a control system, comprising one or more processors, wherein the control system is configured to perform operations comprising providing, as an input to a neural network (NN) model, one or more parameters associated with a geochemical process. The operations also include receiving, as an output of the NN model, a predicted state of the geochemical process. Further, the operations include providing, as an input to a physics-based model, the predicted state of the geochemical process. Further still, the operations include determining, as an output of the physics-based model, a discrepancy associated with the predicted state of the geochemical process. Even further, the operations include controlling operation of oil and gas equipment based on the discrepancy.

The system of any preceding clause, wherein the oil and gas equipment comprise CCS equipment.

The system of any preceding clause, further comprising one or more sensors, wherein the control system is configured to determine the discrepancy based on sensor data acquired by the one or more sensors.

The system of any preceding clause, wherein the predicted state is an extrapolated state.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. Moreover, the order in which the elements of the methods described herein are illustrated and described may be re-arranged, and/or two or more elements may occur simultaneously. The embodiments were chosen and described in order to best explain the principals of the disclosure and its practical applications, to thereby enable others skilled in the art to best utilize the disclosure and various embodiments with various modifications as are suited to the particular use contemplated.

Finally, the techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

1. A method, comprising:
providing, as an input to a neural network (NN) model, one or more parameters associated with a geochemical process;
receiving, as an output of the NN model, a predicted state of the geochemical process;
providing, as an input to a physics-based model, the predicted state of the geochemical process;
determining, as an output of the physics-based model, a discrepancy associated with the predicted state of the geochemical process; and
updating the NN model based on the discrepancy.

2. The method of claim 1, further comprising determining an updated predicted state of the geochemical process using the updated NN model.

3. The method of claim 1, further comprising adjusting operation of one or more oil and gas equipment based on the updated NN model in real-time.

4. The method of claim 1, wherein the geochemical process is a carbon capture and storage (CCS) process.

5. The method of claim 1, wherein the predicted state is associated with CCS precipitation.

6. The method of claim 1, wherein the geochemical process relates to a plume emission.

7. The method of claim 1, wherein the physics-based model utilizes a minimization of Gibbs free energy as a loss function.

8. The method of claim 1, wherein the physics-based model utilizes law of mass action as a loss function.

9. A tangible, non-transitory, machine-readable medium comprising instructions that, when executed by a processor, cause the processor to perform operations comprising:
providing, as an input to a neural network (NN) model, one or more parameters associated with a geochemical process;
receiving, as an output of the NN model, a predicted state of the geochemical process;
providing, as an input to a physics-based model, the predicted state of the geochemical process;
determining, as an output of the physics-based model, a discrepancy associated with the predicted state of the geochemical process; and
updating the NN model based on the discrepancy.

10. The tangible, non-transitory, machine-readable medium of claim 9, wherein the instructions, when executed, further cause the processor to perform the operations comprising controlling one or more oil and gas equipment based on the updated NN model.

11. The tangible, non-transitory, machine-readable medium of claim 10, wherein the one or more oil and gas equipment comprise fluid control devices.

12. The tangible, non-transitory, machine-readable medium of claim 11, wherein the instructions that cause the processor to control the one or more oil and gas equipment comprise adjusting a flow rate provided by the fluid control devices.

13. The tangible, non-transitory, machine-readable medium of claim 11, wherein the instructions that cause the processor to control the one or more oil and gas equipment comprise:
determining a time period to inject one or more fluids within a wellbore; and
outputting a control signal that causes the fluid control devices to inject one or more fluids into a wellbore in accordance with the time period.

14. The tangible, non-transitory, machine-readable medium of claim 11, wherein the one or more parameters associated with the geochemical process relate to a concentration of a plume.

15. The tangible, non-transitory, machine-readable medium of claim 9, wherein the predicted state is an extrapolated state.

16. The tangible, non-transitory, machine-readable medium of claim 9, wherein the instructions that cause the processor to determine the discrepancy based on sensor data and an output of physics-based model.

17. A system, comprising:
one or more downhole tools;
a control system, comprising one or more processors, wherein the control system is configured to perform operations comprising:
providing, as an input to a neural network (NN) model, one or more parameters associated with a geochemical process;
receiving, as an output of the NN model, a predicted state of the geochemical process;
providing, as an input to a physics-based model, the predicted state of the geochemical process;
determining, as an output of the physics-based model, a discrepancy associated with the predicted state of the geochemical process; and
controlling operation of oil and gas equipment based on the discrepancy.

18. The system of claim 17, wherein the oil and gas equipment comprise CCS equipment.

19. The system of claim 17, further comprising one or more sensors, wherein the control system is configured to determine the discrepancy based on sensor data acquired by the one or more sensors.

20. The system of claim 17, wherein the predicted state is an extrapolated state.
